(19) United States
(12) Patent Application Publication
(10) Pub. No.: US 2006/0034824 A1
(43) Pub. Date: Feb. 16, 2006

(54) Pharmaceutical composition comprising carriers for products

(76) Inventors: Cristiano Alberto Ribeiro Santana, Sao paulo (BR); Gilberto De Nucci, Campinas (BR)

Correspondence Address: STITES & HARBISON PLLC, 1199 NORTH FAIRFAX STREET, SUITE 900, ALEXANDRIA, VA, 22314, US

(21) Appl. No.: 10/528,071

(22) PCT Filed: Sep. 19, 2002
(86) PCT No.: PCT/BR02/00133

§ 371 Date: Mar. 17, 2005

(51) Int. Cl. A61K 38/46

(52) U.S. Cl. 424/94.65

(57) ABSTRACT

The present patent application refers to a new product composition comprising: BROMELINE . . . more than 0.01%.

The present invention refers to a new composition pharmaceutical composition comprising carriers for products. Said composition is, preferably, of topical application, non-toxic, features a high penetration rate through the skin, being presented in the form of a gel, cream, gel-cream, aerosol, spray, liquid, lyophilized, patch (transdermal adhesive) or any other dermal or transdermal application.

### BRIEF DESCRIPTION OF THE INVENTION

The skin permeability varies according to the region of the body, being the skin folds and the face those that present the highest absorption rate.

According to the classic book “Histologia dos epitélios”, by Walter A. Hadler and Sineli R. Silveira, Editora Campus, Campinas, 1993, it is considered that: “bearing in mind the general morphological characteristics and the specialized functions that they perform, the epithelium cells are predominantly classified into two categories, which correspond to two epithelium classes: coating epithelium cells and secreting epithelium cells. The cells of these two classes mix with each other to constitute, respectively, the coating epithelia and the secreting epithelia, each one of them performing specific functions that are inherent to them. Such division is also based in the distribution of these two classes of epithelium in the organism, which although wide is distinctive for both. With the purpose of forming the coating epithelia the epithelium cells associate side-by-side, so as to originate “membranes” or layers superimposed over the base membrane, which function is to coat surfaces. On the contrary, the secreting cells unite to form organized functional units, better suited for performing their specialized function, related to the secretion products synthesis; thus are constituted the secreting units. The coating epithelia are defined as living membranes, usually featuring a discontinuity, that isolate the organism from the environment, separating the internal media from the external one. Furthermore, these epithelia isolate from each other the various internal media compartments, among which are the intravascular compartment, the serum compartment and several others. Among the various functions performed by the coating epithelia some are performed by specialized variants that are specifically adapted to perform one or more functions. Others are incorporated as general functions presented without distinction by every coating epithelium cell. The coating epithelium cell, in the same way as most of the living cells, passively absorbs water and electrolytes and eliminates them actively; this function is well developed in the epithelium cells. On that account it is very important to observe that generally it is understood as absorption the penetration of solutions through the cells plasmatic membrane. However two different specific forms of absorption must be distinguished from one another: the passive absorption, that occurs according to the osmotic laws, and the active absorption, that entails the effective participation of the epithelium cell and that does not follow such physic laws. On the other hand it must be considered that every single substance that penetrates the interior of a multi-cellular organism, or else is excreted or eliminated, must cross at least one coating epithelium, because every superior organism is penetrated internally and externally by epithelia. It must also be observed that the coating epithelia, although continuously covering and protecting those surfaces it coats, are not impervious at all; hat is why they do not behave as inert “membranes”. On the contrary, they allow for the exchange of gases, water, several kinds of electrolytes and certain other solutes between the internal and the external media, or between the various internal compartments, which characterizes its permeability. The coating epithelium cells limit in a controlled and selective way the permeability of the respective epithelia, with the purpose of protecting the organism and still participate of the control of its homeostasis. In order to perform such function the epithelia are organized and arrange their cells in a special form, in order to build up coatings which cells abut the base membrane and are united with each other by means of intracellular junctions; in turn the cells are coated by the plasmatic membrane, which features special characteristics, and by the glycocup, both able to express well defined functional properties. The functional characteristics expressed by the plasmatic membrane portion that coats the cells apical surface are different from those expressed by the portion situated in its basal or basolateral face; such differences, which occur mainly on the functional aspect, contribute for the remarkable degree of polarization expressed by the coating epithelium cells. The prime function performed by the coating epithelia correspond essentially to the protection rendered to the surface that they coat, characterizing their protective coating function. Such function features a special characteristic, being a coating that, besides offering mechanical, physical and chemical protection to the coated surface, is not inert. The coating epithelia are pervious, which allows for the controlled and selective passage of several products through its wall. There are many evidences in favor of the idea that the coating epithelia permeability constitutes a fundamental property, with significant functional expression, for it is essential for the performance of several functions featured by the epithelia, even more so because it is selective and its permeability degree presents a wide variation. It is fairly well demonstrated that the permeability degree influences strongly the function performed by the coating epithelia:

1) wide permeability;

2) reduced permeability and

3) absence of permeability.

When there is a wide permeability, the epithelia allow intense metabolic exchanges through their walls, with poor control and selectivity of its permeability. In these circumstances the epithelium acts on the filtration and transfer of metabolites, these functions requiring little qualitative control; the exercise of these functions is subordinated to the epithelium intrinsic structure, which is adapted to act, mainly passively, being low the level of selective permeability.

The coating epithelia with a reduced degree of permeability, due to the characteristic that is so peculiar to them, present the property of partially controlling their own permeability, and above all their selectivity. As a consequence, these coating epithelia present selective permeability, which allows them to interfere and qualitatively control their functional activity, as well as making them more able to actuate over the homeostasis control. The absence of epithelium permeability is correlated to the complex isolation of the coated surface and, on the other hand, to the better controlling of this epithelium function, because its cells, although very poorly pervious, present selective permeability. In this case the coated surface has its boundaries limited by a “membrane” impervious or very poorly pervious and very effective, that performs an important protective function, for it is able to discriminate exactly what can cross the epithelium. The coating epithelia permeability is such an expressive functional property that it has been used as an important classification criterion to rank them in three classes:

1) pervious epithelia;

2) poorly pervious epithelia and

3) impervious epithelia.

Because of their selective permeability, even in the inferior animals the epithelia have assumed the function of coating the organism, constituting its external coating, with limiting and protective properties, not only morphological but also functional. Their cells, in principle very similar, behaved as a semi-pervious “membrane” poorly effective that acted passively, but which function allowed the separation, tough precarious and more morphological than functional, between the internal and the external media. It seem to be that the majority of the coating epithelia acts as a barrier that prevents the free passive diffusion, because their permeability, which is selective, is conditioned to several factors among which stands out the electric potential present in their cell's plasmatic membrane. The continuity of the epithelium coating is established as much through the intimate abutment of adjacent cells as through the presence of intercellular union devices. The epithelium cells are enveloped by the glycocup, that also takes part of the coating function performed by the epithelium, in addition to aid the union between adjacent cells, because the intracellular adhesive is formed also by glycocup. Several experimental investigations confirm that the coating epithelia selective permeability is associated to other specific functions expressed by their cells, namely: absorption, excretion and secretion. These functions, beyond their permeability that constitutes their prime function, are responsible by the general functioning of the epithelium cell. The general functions performed by the coating epithelia are basically the following:

1) surfaces protective coating function;

2) isolation and functional individualization of the internal media and of its distinct compartments, due to their cells selective permeability;

3) controlling the homeostasis of the internal medium and its compartments due to their cells ability to interfere in the epithelium selective permeability; the epithelium cells manifest the capacity to effect the absorption, secretion and excretion; such functions interfere on the epithelium permeability;

4) performance of the metabolic functions due to their ability to effect hydrosaline exchanges and to effect metabolites transfers due to their cells and intracellular spaces high degree of poorly selective permeability;

5) transportation of products along the epithelial surface due to the participation of the cilium;

6) sensorial perception and

7) germinative function.

Among these functions, the first four derive mostly from the epithelium cells selective permeability, over which are additionally superimposed the additional affects corresponding to their properties of absorption, excretion and secretion. Among the general functions performed by the coating epithelia, the selective permeability is responsible by the efficiency regarding the ability to coat, protect and isolate the surfaces, as well as to effect the control of the homeostasis; the passive absorption and the metabolites transfer capacity are executed normally by the majority of the cells of these epithelia, which demand only minor adaptations to become able to effectively perform such functions. On the contrary, the functions of absorption, excretion and secretion depend of properties that develop successively and would become paramount, mostly in some specialized types of coating epithelium, which adapted following a new and specific direction. The sensorial perception and the germinative function are more specific functions that are only manifest by certain epithelia even more specialized. Considering their cell's morphological characteristics, the coating epithelia have been classified according to the same number of cellular extracts they bear in: simple (a single extract) and stratified (two or more extracts). Both the simple epithelia and the stratified ones, conforming to their cells format, are in turn subdivided into pavementous, cubic or prismatic. The simple epithelia are usually adapted to manifest wholly their most expressive fundamental property, which consists in their permeability, which degree and selectivity vary. The simple coating epithelia, constituted by a single layer of pavementous or cubic-prismatic cells, present major differences regarding their functional properties, correlated not only to their cell's morphology, but also to the intracellular space's properties. The simple pavementous epithelia are usually very pervious; the cubic-prismatic ones are less pervious. The coating epithelia permeability, in addition to being selective, is controlled by their cell's functional activity, although the control looses efficiency in the same order as the intracellular space's permeability increases. The cubic-prismatic epithelia, being less pervious than the pavementous, are more effective to control their permeability. Based on the format of the epithelium cell, in its permeability and the coating epithelia most common adaptations, it is possible to generate a provisional classification for these epithelia. Thus, the simple coating epithelia are divided into two classes: pavementous and cubic-prismatic. Each class is subdivided according to its functional properties in open or pervious epithelia, in semi-occlusive or poorly pervious and occlusive or impervious. In the simple coating epithelia classification, the cubic epithelia and the prismatic epithelia are usually considered distinct, being defined and identified according to the format of the epithelium cells that make them up. However some functional studies have showed that the correlation between form and function presents several exceptions. For this reason a functional classification is adopted considering predominantly it's permeability. According to this criterion these epithelia are denominated cubic-prismatic comprising the semi-occlusive and occlusive epithelia. Following the same criterion the stratified epithelia can be subdivided into: pavementous and cubic-prismatic The stratified epithelia are adapted to perform primarily the mechanical protection function, because they are impervious or poorly pervious. The epithelia comprise, in addition to the cells, the intercellular space and the base membrane, which interfere in their permeability degree; their permeability derives not only from their cell's peculiar properties, responsible for the transcellular permeability way, but also from the presence of another permeability way of their walls, constituting the intercellular or paracellular way. The transcellular way comprises two different ways that consist of the transmembranous way and the transcanicullar or trancitose way. It has been demonstrated, experimentally, that the coating epithelia can be transposed by water and by substances of various natures, both through their epithelium cells (transcellular way) and through the way situated between their cells (intercellular way). In the first instance the epithelium cell can effect the permeability control of the epithelium through its biological activity, making this process selective. As for the intercellular way permeability, the epithelium cell, although not behaving in a totally passive form, does not interfere directly in the transport selectivity. The sole form of cell active participation, in this instance, comprises the determination, exceptionally; the enlargement of the corresponding intercellular space. By means of the action of the microfilaments that constitute its cito-skeleton, the epithelium cell, specially those of certain types of simple coating epithelia pavementous of the open type, can change its format and retract segments of its cytoplasm; thus being able to influence the size of the intercellular space and regulate it. It has been established that the transcellular permeability of the simple coating epithelia is perfectly distinct from the intercellular permeability, because both are subordinated to very different mechanisms. The epithelium cell permeability, which is selective, is influenced by its biological activity; on the contrary, the intercellular permeability is totally passive, and thus is not selective. Several experimental results have confirmed that the transposition of solutions through the epithelia is subject to multiple control mechanisms, among which is paramount the intrinsic functional activity of its cells. On the contrary, the intercellular space permeability is generally not controlled, because in this case the transposition of a molecule through the epithelium follows only the corresponding physical laws and is directly related to its diameter, its electrical cargo and, obviously, to the intercellular space size; these three variables constitute the main limiting factors that interfere on the intercellular permeability of the simple coating epithelia. The transcellular permeability of the simple coating epithelia can be exercised through two distinct and independent ways: the transmembrane way, which is the true transcellular way, and the transcanicullar way, which happens through the vesicles and the cannules or tubes of the vesicle-cannule system, found inside the cytoplasm of many types of coating epithelium cells”. Consequently, the coating epithelia are pervious, which allows the controlled and selective passage of various products through its wall. It is demonstrated that the permeability degree affects strongly the coating epithelia function.

Three types of coating epithelia are thus considered:

1—Of wide permeability;

2—Of reduced permeability;

3—Of null permeability.

The purpose is to prove through the formulation that there is an intense metabolic exchange demonstrating that the epithelium actuates on the transfer of metabolites. This penetration of substances is complete and gradual and trespasses these epithelium layers until it penetrates the small blood vessels, reaching the circulatory current.

There is a description of the molecules to estimate the coating epithelia permeability. Ex.: Hemoglobin, Ferritine, Lipoproteins and enzymes.

It is also known the transcitose on the transposition of the epithelia by the macro and micro molecules until the vascular eye depending of their association.

BROMELINE is the generic name given to the proteolytic enzymes found in pineapple and other species of plants of the BROMELIACEAE family. The bromelines hydrolyze a vast series of proteins, peptides, esters and amides. The enzyme, obtained from the stem, is of glycoproteic nature.

In a way similar to what happens to the papaine, Bromeline presents, in it's mechanism of activation and deactivation, a sulphydrilic protease, that is, the action of its enzymes depends on the sulphydric group of a cisteyne residue Conversely, and different from the papaine, on the hydrolysis of the glucagon the later is cleaved by BROMELINE in different spots.

The presence of an enzyme with notorious proteolytic activity in fresh pineapple juice has been known for a long time.

An enzyme can be isolated from the fruit and all the remaining parts of the plant. It was detected that the BROMELINE's activity in the fruit is higher in some cultures than in others and also in the juicy portions of the fruit. The levels of BROMELINE are relatively high in the juice extracted from the partially ripe fruit, decreasing remarkably with the development of full ripeness.

Some studies have indicated that the resin is not present in the first stages of fruit development, increasing rapidly until the ripening, when a slight decrease is detected.

The sensitive decrease on the protease activity during the final ripening period is not followed by a corresponding change in the protein concentration.

Pineapple is therefore the only fruit presenting relatively high protease concentrations in the ripe stage of the fruit.

In order to produce BROMELINE different parts of the plant are used: leaves, stem, fruit juice, skin, as well as the industrial residues.

It is more frequent to use as raw material the stems of the ripe pineapple bushes, used after the fruits are collected.

BROMELINE occurs in a higher concentration in the lower parts of the stems of ripe plants, and the core portion of it contains more protease than the external part.

Several research studies relate those properties of BROMELINE extracted from the pineapple juice and from its stem. Said studies disclose the existence of differences in the enzymatic properties between the stem and the fruit juice, as well as indicate that the proteolytic activity of the enzyme depends on factors such as the variety, placement, part and age of the plant.

The existence of at least 2 main components in the stem BROMELINE is known, with similar molecular properties and proteolytic activities. In the same way 5 proteolytically active components were separated from a BROMELINE enzymatic preparation with BROMELINE extracted from the stem. Said fractions presented similar results for some parameters and different properties for others.

Said studies disclosed that the raw material contains several proteolytically active components, some differing in their molecular weight and their charge, others being so similar that they could only be fractionated by means of using special conditions.

Normally the BROMELINE is obtained by means of the addition of precipitating agents, such as ketones, alcohols and some salts, among others, to the juices of the stem and the pineapple plant.

In an industrial scale prevails the obtainment of the enzyme from the stem of the pineapple plant, which is a by-product derived from the plant's exploitation.

The literature presents patents for the obtainment of the raw enzyme from the stem. Said patents teach the extraction of bromeline through precipitation with organic solvents and water-soluble salts.

Some patents describe the path for obtainment of BROMELINE through the saline precipitation of the enzyme retrieving it by means of an agglutinating organic agent.

The commercial value and quality of the bromeline are intimately associated to its proteolytic activity. Therefore there are several different methods to gauge the BROMELINE activity through its proteolytic activities, such as its catalytic potential in the hydrolysis of either proteic or synthetic substrates.

Also of common knowledge are its uses in the processing of leather, in the food and drink processing industry, the textile industry and the treatment of internal disturbances of the digestive system.

However its uses and applications up to now have excluded the dermatological forms, in view of its instability and degradability.

The Applicant thus achieved the use of bromeline in dermatological applications in a surprising and unexpected way, achieving penetration rates and absorption of the active ingredient that proved to be much more expressive than the ones expected for these applications. The applicant achieved said object by associating the composition to BROMELINE according to the present invention.

The object of the present invention is thus a new composition of the carrier substance for products containing BROMELINE.

Advantageously said composition comprises:

| | |
|---|---|
| BROMELINE | more than 0.01%. |

More advantageously the composition comprises:

| | |
|---|---|
| BROMELINE | from 2 to 5%. |

Particularly the composition of the invention comprises:

| | |
|---|---|
| BROMELINE | more than 0.01% |
| HYALURONIDASE | 50 to 900 utr/mg. |

Advantageously the present composition comprises:

| | |
|---|---|
| BROMELINE | more than 0.01% |
| HYALURONIDASE | 50 to 900 utr/mg |
| VITAMINE E | 10 to 2000 mg |

More particularly the composition of the present invention comprises:

| | |
|---|---|
| BROMELINE | more than 0.01% |
| HYALURONIDASE | 250 to 350 utr/mg. |

Even more particularly the composition of the present invention
comprises:

| | |
|---|---|
| BROMELINE | de 2 to 5% |
| HYALURONIDASE | 250 to 350 utr/mg. |

Advantageously the present composition comprises:

| | |
|---|---|
| BROMELINE | more than 0.01% |
| PAPAINE | more than 0.01%. |

Even more advantageously the present composition comprises:

| | |
|---|---|
| BROMELINE | more than 0.01% |
| PAPAINE | more than 0.01% |
| HYALURONIDASE | 50 to 900 utr/mg. |

Even more advantageously the present composition comprises:

| | |
|---|---|
| BROMELINE | more than 0.01% |
| PAPAINE | more than 0.01% |
| VITAMINE E | 10 to 2000 mg |
| HYALURONIDASE | 50 to 900 utr/mg. |

Advantageously the present composition comprises:

| | |
|---|---|
| BROMELINE | 2 to 5% |
| PAPAINE | more than 0.01%. |

More advantageously the present composition comprises:

| | |
|---|---|
| BROMELINE | more than 0.01% |
| PAPAINE | 2 to 5%. |

Even more advantageously the present composition comprises:

| | |
|---|---|
| BROMELINE | more than 0.01% |
| PAPAINE | 2 to 5% |
| HYALURONIDASE | 50 to 900 utr/mg. |

Even more advantageously the present composition comprises:

| | |
|---|---|
| BROMELINE | 2 to 5% |
| PAPAINE | more than 0.01% |
| HYALURONIDASE | 50 to 900 utr/mg. |

According to the present invention the composition may be presented in the form of a gel, cream or gel-cream, liquid, spray, aerosol, lyophilized, patch (transdermal adhesive) or any other dermal or transdermal form.

Said composition may be applied either dermically or transdermically.

The present composition may be used in the production de medicines.

The present invention was tested by means of studies in 24 outpatients, in three different sections with groups of 8 outpatients, being the concentration higher than 0.01%.

The area delimited was 15 cm×10 cm (150 cm²), with the application of cream. After 15 minutes the measurements were initiated, through liquid chromatography, coupled to mass spectrophotometry.

Some of the substances that can be carried are presented bellow with a merely illustrative, non-limiting purpose:
CUTANEOUS CICATRIZATORS
PURE ANTIBIOTICS AND SULFA DERIVATIVES
TOPICAL DERMATOLOGIC ANTI-FUNGUS AGENT
TOPICAL RUBIFACIENT ANTIREUMATICS
CORTICOSTEROIDS
ANTIMICOTICS
PURE AND ASSOCIATED ANTIBACTERICIDES
TOPICAL ANTI-VARIX
ANTI-HISTAMINIC ANTI-ITCH
TOPICAL ANTIVIRALS
TOPICAL LOCAL ANESTHETICS
HORMONAL AND NON-HORMONAL ANTI-INFLAMMATORY
HISTAMINE CLOROHYDRATE
FILDENAFIL CITRATE
FENTOLAMINE MESILATE
PROSTAGLANDINE

1. Pharmaceutical composition comprising carriers for products characterized by the fact that it comprises:
Bromeline . . . more than 0.01% .

2. Pharmaceutical composition comprising carriers for products characterized by the fact that it comprises:
Bromeline . . . 2 to 5%.

3. Pharmaceutical composition comprising carriers for products according to claim 1, characterized by the fact that it comprises:
Bromeline . . . more than 0.01%.
Hyaluronidase . . . 50 to 900 utr/mg.

4. Pharmaceutical composition comprising carriers for products according to claim 1, characterized by the fact that it comprises:
Bromeline . . . more than 0.01%
Hyaluronidase . . . 50 to 900 utr/mg
Vitamine E . . . 10 to 2000 mg

5. Pharmaceutical composition comprising carriers for products according to claim 1, characterized by the fact that it comprises:
Bromeline . . . more than 0.01%
Papaine . . . 0.01 to 15%

6. Pharmaceutical composition comprising carriers for products according to claim 1, characterized by the fact that it comprises:
Bromeline . . . more than 0.01%
Papaine . . . more than 0.01%
Hyaluronidase . . . 50 to 900 utr/mg

7. Pharmaceutical composition comprising carriers for products according to claim 1, characterized by the fact that it comprises:
Bromeline . . . more than 0.01%
Papaine . . . more than 0.01%
Vitamine-E . . . 10 to 2000 mg

8. Pharmaceutical composition comprising carriers for products according to claim 1, characterized by the fact that it comprises”:
Bromeline . . . more than 0.01%
Papaine . . . more than 0.01%
Hyaluronidase . . . 50 to 900 utr/mg
Vitamine E . . . 10 to 2000 mg

9. Pharmaceutical composition comprising carriers for products according to claim 1, characterized by the fact that it comprises:
Bromeline . . . 2 to 5%
Papaine . . . more than 0.01%.

10. Pharmaceutical composition comprising carriers for products according to claim 1, characterized by the fact that it comprises:
Bromeline . . . more than 0.01%
Papaine . . . 2 to 5%.

11. Pharmaceutical composition comprising carriers for products according to claim 1, characterized by the fact that it comprises:
Bromeline . . . more than 0.01%
Papaine . . . 2 to 5%
Hyaluronidase . . . 50 to 900 utr/mg.

12. Pharmaceutical composition comprising carriers for products according to claim 1, characterized by the fact that it comprises:
Bromeline . . . 2 to 5%
Papaine . . . more than 0.01%
Hyaluronidase . . . 50 to 900 utr/mg.

13. Pharmaceutical composition comprising carriers for products according to claim 1, characterized by the fact that it comprises:
Bromeline . . . 2 to 5%.
Hyaluronidase . . . 50 to 900 utr/mg.

14. Pharmaceutical composition comprising carriers for products according to claim 1, characterized by the fact that it comprises:
Bromeline . . . 2 to 5%.
Hyaluronidase . . . 250 to 350 utr/mg

15. Pharmaceutical composition comprising carriers for products according to claim 1, characterized by the fact that it is presented in the form of a gel, cream, gel-cream, liquid, spray, aerosol, lyophilized, patch (transdermal adhesive) or any other dermal or transdermal form.

16. Pharmaceutical composition comprising carriers for products according to claim 15, characterized by the fact that said composition is applied either dermically or transdermically.

17. Pharmaceutical composition comprising carriers for products according to claim 1, characterized by the fact that said composition is used for the production of a medicine.

18. Pharmaceutical composition comprising carriers for products according to claim 2, characterized by the fact that it is presented in the form of a gel, cream, gel-cream, liquid, spray, aerosol, lyophilized, patch (transdermal adhesive) or any other dermal or transdermal form.

19. Pharmaceutical composition comprising carriers for products according to claim 18, characterized by the fact that said composition is applied either dermically or transdermically.

20. Pharmaceutical composition comprising carriers for products according to claim 2, characterized by the fact that said composition is used for the production of a medicine.
